(19) United States
(12) Patent Application Publication
(10) Pub. No.: US 2014/0264067 A1
(43) Pub. Date: Sep. 18, 2014

(54) AIR CONDITIONING DEVICE

(71) Applicant: Takashi Nakabayashi, Osaka-shi (JP)

(72) Inventor: Takashi Nakabayashi, Osaka-shi (JP)

(73) Assignee: SHARP KABUSHIKI KAISHA, Osaka-shi, Osaka (JP)

(21) Appl. No.: 14/130,663

(22) PCT Filed: Jun. 12, 2012
(86) PCT No.: PCT/JP2012/064990

§ 371 Date: Jan. 2, 2014

(30) Foreign Application Priority Data
Jul. 6, 2011 (JP) 2011-150219

(51) Int. Cl. A61L 9/22

(52) U.S. Cl. CPC A61L 9/22
USPC 250/423 R

(57) ABSTRACT

The air conditioning device discharges ions into air inflowing through an inflow port and outflows the air from the outflow port. The air conditioning device includes an air passage branched into a first passage connected to a peripheral region including a part of a space close to an inner surface of the outflow port and a second passage connected to a central region including a center of the outflow port. The first passage is narrowed continuously from the upstream to the downstream of the air flow, so that the air passing through the first passage is accelerated to outflow at a higher speed than the air passing through the second passage. The ions contained in the air outflowing while passing through the second passage are blocked from charged things around the outflow port by the high-speed air outflowing while passing through the first passage.

This application is the national phase under 35 U.S.C. §371 of PCT International Application No. PCT/JP2012/064990 which has an International filing date of Jun. 12, 2012 and designated the United States of America.

### BACKGROUND

1. Technical Field

The present invention relates to an air conditioning device which performs air-conditioning by discharging ions into air.

2. Description of Related Art

Conventionally, an air conditioning device that cleans air by diffusing ions into air has been used. Such an air conditioning device includes an ion generator that generates positive ions H⁺(H₂O)ₘ and negative ions O₂⁻(H₂O)ₙ, and discharges the ions generated by the ion generator into the air. Herein, m and n are arbitrary natural numbers. Ions discharged into the air surround fungi, bacteria, viruses, or the like and adhered thereto in the air, and the fungi, bacteria, viruses, or the like are deactivated by the action of hydroxide radicals which are active species generated by a reaction therebetween during adhesion. Such techniques are disclosed in Japanese Patent Laid-open Publication No. 2003-47651, Japanese Patent Laid-open Publication No. 2002-319472, and Japanese Patent Laid-open Publication No. 2010-055960, for example.

In addition, a technique for installing an air conditioning device under a floor of a building is disclosed in Japanese Patent Laid-open Publication No. 2010-243075. When the air conditioning device is installed under the floor, an outflow port is provided in the floor of the building.

### SUMMARY OF THE INVENTION

In a state where an air conditioning device is installed under the floor of a building, charged substances are disposed near an outflow port of the air conditioning device as in the case in which the floor surface near the outflow port is covered with a carpet or the like. In this case, the space around the outflow port is sometimes electrostatically charged. If the space around of the outflow port is charged, a part of the ions in the air outflowing from the outflow port are neutralized and dissipate, and the quantity of ions discharged into the air from the air conditioning device decreases, so that the air cleaning ability of the air conditioning device is deteriorated.

In consideration of the above-mentioned circumstances, it is an object of the present invention to provide an air conditioning device which can suppress the deterioration in the air cleaning ability using ions by discharging ions in the air without neutralizing a part of the ions.

An air conditioning device according to the present invention is characterized by comprising: an inflow port through which air inflows from outside; an outflow port through which the air outflows to outside; a passage through which the air inflowing from the inflow port passes to the outflow port; and an ion generator discharging ions to the air, which is disposed at a point along the passage excluding both ends, wherein the passage includes: a first passage configured to outflow the air from a part of the outflow port including one portion of a space close to an inner surface of the outflow port; and a second passage configured to outflow the air from the other part of the outflow port, and a speed of the air outflowing through the first passage is higher than a speed of the air outflowing through the second passage.

In the air conditioning device according to the present invention, ions are discharged into an air inflowing from the inflow port and the air containing ions outflows from the outflow port. An air passage includes a first passage which is connected to a part of the outflow port including one portion of a space close to an inner surface of the outflow port, and a second passage which is connected to other portion of the outflow port. The air passing through the first passage outflows at a higher speed than the air passing through the second passage. The ions contained in the air outflowing while passing through the second passage are blocked from electrically charged things around the outflow port by the high-speed air outflowing while passing through the first passage.

The air conditioning device according to the present invention is characterized in that the first passage is narrowed continuously from the upstream to the downstream of the air flow.

The air conditioning device according to the present invention is characterized in that the second passage is widened continuously from the upstream to the downstream of the air flow.

In the present invention, the first passage is narrowed continuously from the upstream to the downstream of the air flow, such that the air passing through the first passage is accelerated gradually, and then outflows.

The air conditioning device according to the present invention is characterized by further comprising: a partition plate disposed inward from the outflow port so as to separate the first passage and the second passage from each other, wherein the partition plate is inclined so as to narrow the first passage from the upstream to the downstream of the air flow.

In the present invention, the partition plate is disposed inward from the outflow port, such that the first passage and the second passage are separated from each other by the partition plate. In addition, the first passage is narrowed from the upstream to the downstream of the air flow by the inclined partition plate.

The air conditioning device according to the present invention is characterized in that the outflow port is disposed on an upper side.

The air conditioning device according to the present invention is characterized in that the passage is branched into the first passage and the second passage at a downstream position from the ion generator.

In the present invention, the outflow port is disposed on the upper side of the air conditioning device, and the air conditioning device is installed under a floor of the building. The air outflowing from the space close to the inner surface of the outflow port while passing through the first passage prevent foreign matters falling from the outflow port.

According to the present invention, the ions contained in the air outflowing through the second path of the air conditioning device are discharged into the air flowing in a space without being neutralized even if the circumference of the outflow port is electrostatically charged. Therefore, the air conditioning device of the present invention is capable of suppressing the deterioration of the air cleaning ability using ions, and stably cleaning the air.

The above and further objects and features of the invention will more fully be apparent from the following detailed description with accompanying drawings.

### BRIEF DESCRIPTION OF THE SEVERAL VIEWS OF THE DRAWINGS

FIG. 1 is a schematic view illustrating a state that an air conditioning device according to the present invention is installed in a building;

FIG. 2 is a typical view illustrating an outer appearance of the air conditioning device;

FIG. 3 is a typical perspective view illustrating a configuration of inside a casing of the air conditioning device;

FIG. 4 is a typical perspective view of an ion generator;

FIG. 5 is a schematic view illustrating the configuration of inside the casing as seen from above;

FIG. 6 is a block diagram illustrating an internal functional configuration of a controller;

FIG. 7 is a typical view illustrating an example of the internal configuration of the air conditioning device during an operation;

FIG. 8 is a typical view illustrating another example of the internal configuration of the air conditioning device;

FIG. 9 is a typical view illustrating another example of the internal configuration of the air conditioning device;

FIG. 10 is a typical view illustrating another example of the internal configuration of the air conditioning device;

FIG. 11 is a typical view illustrating another example of the internal configuration of the air conditioning device;

FIG. 12 is a typical view illustrating another example of the internal configuration of the air conditioning device; and

FIG. 13 is a typical view illustrating another example of the internal configuration of the air conditioning device.

### DETAILED DESCRIPTION

Hereinafter, the present invention will be described in detail with reference to the accompanying drawings illustrating the embodiments thereof.

FIG. 1 is a schematic view illustrating a state that an air conditioning device according to the present invention is installed in a building. An air conditioning device 1 of the present invention forms a part of an air conditioning system and is installed under a floor of a building 5. FIG. 1 illustrates a schematic cross-section of the building 5 as seen from a side of the building 5. The air conditioning device 1 is installed on a lower side from a floor 51 of the building 5 so that the upper surface thereof to is the same level as an upper face of the floor 51. A controller 2 is installed in the building 5 for controlling the operation of the air conditioning device 1. In addition, a blower 3 is installed in the building 5 and the blower 3 and the air conditioning device 1 are connected through a duct 4. The blower 3 blows air into the duct 4. The air sent from the blower 3 flows in the duct 4 to inflow into the air conditioning device 1. The air inflowing into the air conditioning device 1 outflows to a space on the upper side from the floor 51. In FIG. 1, the flow of air is illustrated by arrows.

FIG. 2 is a typical view illustrating an outer appearance of the air conditioning device 1. In the air conditioning device 1, a cover 12 with a net-shaped structure is put on the upper side of a rectangular-shaped casing 11. FIG. 3 is a typical perspective view illustrating a configuration of inside the casing 11 of the air conditioning device 1. The outer appearance of the casing 11 is illustrated by a dashed-two dotted line in FIG. 3. The casing 11 has an air inflow port 13 formed on a bottom surface thereof. The duct 4 is connected to the bottom surface of the casing 11, so that the air flowing in the duct 4 inflows into the casing 11 through the inflow port 13. The casing 11 also has an outflow port 14 formed on an upper surface thereof. The air inflowing into the casing 11 through the inflow port 13 outflows from the outflow port 14. The cover 12 is configured to cover the outflow port 14. When the air conditioning device 1 is installed, the upper surface of the cover 12 is substantially the same level as the surface of the floor 51. Since the cover 12 has a net-shaped structure, it is possible to prevent foreign matters from falling into the casing 11, while passing through the air outflowing from the outflow port 14.

The air conditioning device 1 includes two ion generators 15 and 15 mounted in the casing 11. FIG. 4 is a typical perspective view of an ion generator 15. The ion generator 15 which is formed in a rectangular shape generates H⁺ (H₂O)ₘ of positive ions and O₂⁻ (H₂O)ₙ of negative ions. The ion generator 15 is provided with an ion discharging surface 151 in which negative ion discharging portions 152 and positive ion discharging portions 153 are formed. FIG. 4 illustrates a type in which the ion generator 15 is provided with two negative ion discharging portions 152 and two positive ion discharging portions 153. Herein, a distance between the negative ion discharging portion 152 and the positive ion discharging portion 153 is longer than the distances between the negative ion discharging portions 152 and 152 and the positive ion discharging portions 153 and 153. Two ion generators 15 and 15 are fixed to inner side surfaces of the casing 11 so as to face the ion discharging surfaces 151 thereof each other. The ion generators 15 and 15 are disposed at a point along a passage through which the air inflowing from the inflow port 13 passes to the outflow port 14, excluding both ends of the passage. The ion generators 15 and 15 discharge ions to the air passing therebetween.

Further, the air conditioning device 1 includes partition plates 16 and 16 configured to each branch the air passage from the inflow port 13 to the outflow port 14. Two partition plates 16 and 16 are arranged in a lengthwise direction of the casing 11 together so as to hang obliquely downward from the outflow port 14. FIG. 5 is a schematic view illustrating the configuration of inside the casing 11 as seen from above. The ion generators 15 and 15 and the partition plates 16 and 16 are seen in the outflow port 14, and the inflow port 13 is seen at the back thereof. The partition plates 16 and 16 are disposed inwardly from the ion generators 15 and 15 above the inflow port 13. In addition, each of the partition plates 16 is disposed at a position near to the side surface of the casing 11 from the center of the outflow port 14. Further, each of the partition plates 16 and 16 is connected to the inner surface of the casing 11 through connecting portions 161 so as to be fixed in an internal space of the casing 11. The connecting portions 161 are not shown in FIG. 3. Upper ends of the partition plates 16 and 16 contact the cover 12, while lower ends thereof are positioned in the middle of the internal space of the casing 11. Each of the partition plates 16 is inclined with respect to the inner surface of the casing 11 so as to narrow gradually the distance between the inner surfaces of the casing 11 facing each other toward the outflow port 14. That is, the partition plates 16 are inclined from a vertical direction so that the distance between the partition plates 16 and the inner surfaces of the casing 11 is narrowed gradually toward the outflow port 14.

FIG. 6 is a block diagram illustrating an internal functional configuration of the controller 2. The controller 2 is provided with a control unit 21 including a processing unit configured to perform a processing for controlling the air conditioning device 1 and a storage unit configured to store data necessary for the processing. The control unit 21 is connected with an operation unit 22 including operation buttons configured to receive operation from a user. In addition, the control unit 21 is connected with a display unit 23 configured to display information necessary for operation. The display unit 23 may employ a liquid crystal panel, for example. The operation unit 22 and the display unit 23 may be formed by a touchpad. Further, the control unit 21 is connected with an external power supply 20 such as a commercial power supply or the like, which supplies power to the control unit 21. Furthermore, the control unit 21 is connected with the ion generators 15 and 15 of the air conditioning device 1. The ion generators 15 and 15 and the control unit 21 are connected through a side wall of the casing 11.

When the operation unit 22 receives an instruction of operation start input from the operation unit 22 operated by a user, the control unit 21 controls to start the operation of the ion generators 15 and 15, and the display unit 23 displays information showing the condition in operation of the air conditioning device 1. When the operation unit 22 receives an instruction of operation stop, the control unit 21 controls to stop the operation of the ion generators 15 and 15. In addition, the controller 2 may be provided with a power supply therein. Further, the control unit 21 may be provided in the air conditioning device 1. In this embodiment, the operation unit 22 and the display unit 23 included in the controller 2 may be connected with the control unit 21 included in the air conditioning device 1.

Next, the operation of the air conditioning device 1 will be described. FIG. 7 is a typical view illustrating the inside the air conditioning device during operation. FIG. 7 illustrates the configuration of inside the air conditioning device 1 as seen from the front, while showing a cut away cross section of the casing 11. The air sent from the blower 3 passes through the duct 4 to inflow into the air conditioning device 1 from the inflow port 13. The air inflowing from the inflow port 13 flows to the outflow port 14. The ion generators 15 and 15 are disposed in the middle of the air passage from the inflow port 13 to the outflow port 14. The ion generators 15 and 15 discharges ions to the air flowing along the ion discharging surface 151. As a result, the air containing ions flows toward the outflow port 14. In FIG. 7, the flow of air is illustrated by arrows, while the negative ions and the positive ions are illustrated by − and + in circles, respectively.

An air passage downstream from the ion generators 15 and 15 of the air passage from the inflow port 13 to the outflow port 14 is branched to the first passages 17 and 17 and a second passage 18 by the partition plates 16 and 16. The first passage 17 is a space sandwiched between each of the partition plates 16 and the inner surface of the casing 11, and the second passage 18 is a space sandwiched between two partition plates 16 and 16. Since a distance between each of the partition plates 16 and the inner surface of the casing 11 is narrowed gradually toward the outflow port 14, the first passage 17 is narrowed continuously from the upstream to the downstream of the air flow. In addition, two partition plates 16 and 16 are slantly disposed toward each other so as to widen the distance there between toward the outflow port 14, and the second passage 18 is continuously extended from the upstream to the downstream of the air flow. Therefore, the air passing through the first passage 17 is accelerated gradually, while the air passing through the second passage 18 is gradually decelerated.

In the outflow port 14, the air passing through the first passages 17 outflows from a peripheral region 141 including the space close to the inner surface of the outflow port 14, while the air passing through the second passage 18 outflows from a central region 142 including the center of the outflow port 14. Since the air passing through the first passages 17 is accelerated, and the air passing through the second passage 18 is decelerated, the air outflowing from the peripheral region 141 of the outflow port 14 flows at a higher speed than the air outflowing from the central region 142 thereof. In FIG. 7, the speed of air outflowing from the outflow port 14 is indicated by the length of arrows.

The ions contained in the air outflowing from the peripheral region 141 of the outflow port 14 eliminate charges from electrically charged things around the outflow port 14. On the other hand, the ions contained in the air outflowing from the central region 142 of the outflow port 14 are blocked from electrically charged things around the outflow port 14 by the high-speed air outflowing from the peripheral region 141. Therefore, the ions contained in the air outflowing from the central region 142 of the outflow port 14 are discharged without being neutralized to therefore discharge into the air of a space in the building 5. The air of the space in the building 5 is cleaned by the discharged ions. In this way, even in the electrically charged-state around the outflow port 14, the air is stably cleaned. Therefore, in the air conditioning device 1 according to the present embodiment, it is possible to stably clean the air by suppressing the deterioration of the air cleaning ability using ions. In addition, in the present embodiment, since the air outflows from the peripheral region 141 of the outflow port 14 at a high speed, falling of foreign matter from around the outflow port 14 into the air conditioning device 1 may be suppressed.

Next, other configuration examples of the air conditioning device 1 will be described. FIGS. 8 to 13 are typical views illustrating examples of the internal configuration of the air conditioning device 1. Each drawing illustrates the inside of the air conditioning device 1 as seen from the front, while showing the cut away cross section of the casing 11. In addition, the flow of air is illustrated by arrows in each drawing. FIG. 8 illustrates an air conditioning device 1 including one ion generator 15. The ion generator 15 is mounted on one inner surface of the casing 11. In this embodiment, the air containing the ions generated by the ion generator 15 outflows at a speed accelerated by passing through the first passages 17 and 17, and outflows at a speed decelerated by passing through the second passage 18.

FIG. 9 illustrates an air conditioning device 1 including one partition plate 16. The air passage is branched into a first passage 17 and a second passage 18 by one partition plate 16. Also in this embodiment, the air containing the ions generated by the ion generators 15 and 15 outflows at a speed accelerated by passing through the first passages 17 and 17, and outflows at a speed decelerated by passing through the second passage 18. According to this embodiment, in the outflow port 14, the peripheral region 141 includes only one portion of the space close to the inner surface of the outflow port 14 as seen from the front, and the central region 142 includes the other portion of the space close to the inner surface of the outflow port 14. When the air conditioning device 1 is disposed at the end of the floor 51, electrically charged objects are rarely disposed in the vicinity of a portion of an inner edge of the outflow port 14 near the end of the floor 51. Therefore, when the air conditioning device 1 is disposed in such way that the portion of the space close to the inner surface of the outflow port 14 included in the central region 142 is closer to the end of the floor 51 than the portion included in the peripheral region 141, an effect of discharging the ions from the central region 142 without being neutralized may be obtained.

FIG. 10 illustrates an air conditioning device 1 including one partition plate 16 and ion generators 15 provided in the middle of each of a first passage 17 and a second passage 18. In this embodiment, the ion generators 15 are disposed in the middle of each of the first passage 17 and the second passage 18, without branching the air passage downstream from the ion generators 15. The ions from the ion generator 15 are discharged into the air passing through the first passages 17, and the air including the ions outflows at a speed accelerated. On the other hand, the ions from the ion generator 15 are discharged into the air passing through the second passage 18, and the air including the ions outflows at a speed decelerated.

FIGS. 11 and 12 illustrate an air conditioning device 1 including one partition plate 16 and one ion generator 15, respectively. More specifically, the air conditioning device 1 shown in FIG. 11 includes an ion generator 15 mounted on the inner surface of the casing 11 near a second passage 18, while the air conditioning device 1 shown in FIG. 12 includes an ion generator 15 mounted on the inner surface of the casing 11 near a first passage 17. Also in these embodiments, the air containing the ions generated by the ion generators 15 outflows at a speed accelerated by passing through the first passage 17, and outflows at a speed decelerated by passing through the second passage 18.

FIG. 13 illustrates an air conditioning device 1 including a casing 11 which is formed in such a way that the distance between the inner surfaces of the casing 11 is narrowed continuously from the upstream side to the downstream side of the air flow. FIG. 13 illustrates an example in which the external appearance of the casing 11 corresponds to the inner surface thereof, but the casing 11 may have a shape in which the outer surface does not correspond to the inner surface thereof. In addition, two partition plates 16 and 16 are disposed in the casing 11 so as to hang vertically downwardly from the outflow port 14 together. A second passage 18 sandwiched between the two vertical partition plates 16 and 16 has a space which is not variable from the upstream to the downstream of the air flow. While employing the vertical partition plates 16 and 16, but the distance between the inner surfaces of the casing 11 is narrowed continuously from the upstream side to the downstream side of the air flow. That is, since the inner surfaces of the casing 11 are inclined radially inward in a vertical direction with respect to the vertical partition plates 16 and 16, first passages 17 and 17 sandwiched between the inner surfaces of the casing 11 and the partition plates 16 and 16 are narrowed continuously from the upstream to the downstream of the air flow. Therefore, the air passing through the first passages 17 and 17 is accelerated gradually. Thereby, also in this embodiment, the air containing the ions generated by the ion generators 15 outflows at a speed accelerated by passing through the first passages 17, and outflows at a speed decelerated by passing through the second passage 18.

As mentioned above, in all embodiments illustrated in FIGS. 8 to 13, the air outflowing from the peripheral region 141 of the outflow port 14 passing through the first passage 17 outflows at a higher speed than the air outflowing from the central region 142 of the outflow port 14. Therefore, the ions contained in the air outflowing from the central region 142 of the outflow port 14 are blocked from something around the outflow port 14 by the high-speed air outflowing from the peripheral region 141 without being neutralized to therefore discharge into the air of the space in the building 5. Accordingly, the air of the space in the building 5 is stably cleaned.

In addition, the present embodiment illustrates the configuration that the air passage is branched into the first passage 17 and the second passage 18 by the partition plates, but the present invention is not limited thereto, and may have a configuration that the air conditioning device 1 includes a first passage 17 and a second passage 18 which are formed by such a way that a partition plate is integrally formed with a casing 11 or the like. Further, the present embodiment illustrates the configuration in which the air passage is branched into the first passage 17 and the second passage 18, but the present invention may have a configuration in which the air conditioning device 1 includes an air passage from the inflow port 13 to the outflow port 14 which is completely separated into a first passage 17 and a second passage 18. In the modified embodiment in which the air passage is completely separated into the first passage 17 and the second passage 18, ion generators 15 are disposed in the middle of each of the first passage 17 and the second passage 18. Further, in the present embodiment, the first passage 17 and the second passage 18 are partitioned by one plate, but the present invention is not limited thereto, and may have another configuration such as a configuration in which the whole circumference of the space close to the inner surface of the outflow port 14 is provided in the peripheral region 141 or the like.

In addition, the present embodiment illustrates the configuration in which the inflow port 13 is formed on the bottom surface of the casing 11, but the air conditioning device 1 may have a configuration in which the inflow port 13 is formed on a side surface of the casing 11. Further, the present embodiment illustrates the configuration in which the blower 3 is installed on the outside of the air conditioning device 1, but the air conditioning device 1 may have a configuration including a blower unit such as a fan or the like therein. Furthermore, the present embodiment illustrates the configuration in which the outflow port 14 is formed on the upper side of the air conditioning device 1 which is provided under the floor, but the present invention is not limited to the configuration of installing the air conditioning device 1 under the floor. For example, the air conditioning device 1 may have a configuration in which an inflow port 13 is formed on the upper side thereof and an outflow port 14 is formed on the bottom side thereof, wherein the air conditioning device 1 is provided in the ceiling of the building 5. In this embodiment, the air conditioner 1 also may stably clean the air in the electrically charged-state around the outflow port 14.

As this invention may be embodied in several forms without departing from the spirit of essential characteristics thereof, the present embodiment is therefore illustrative and not restrictive, since the scope of the invention is defined by the appended claims rather than by the description preceding them, and all changes that fall within metes and bounds of the claims, or equivalence of such metes and bounds thereof are therefore intended to be embraced by the claims.

1-4. (canceled)

5. An air conditioning device, comprising:
an inflow port through which air inflows from outside;
an outflow port through which the air outflows to outside;
a passage through which the air inflowing from the inflow port passes to the outflow port; and
an ion generator discharging ions to the air, which is disposed at a point along the passage excluding both ends,
wherein the passage includes:
a first passage configured to outflow the air from a part of the outflow port including one portion of a space close to an inner surface of the outflow port; and
a second passage configured to outflow the air from the other part of the outflow port, and
a speed of the air outflowing through the first passage is higher than a speed of the air outflowing through the second passage.

6. The air conditioning device according to claim 5,
wherein the first passage is narrowed continuously from the upstream to the downstream of the air flow.

7. The air conditioning device according to claim 6,
wherein the second passage is widened continuously from the upstream to the downstream of the air flow.

8. The air conditioning device according to claim 6, further comprising:
a partition plate disposed inward from the outflow port so as to separate the first passage and the second passage from each other,
wherein the partition plate is inclined so as to narrow the first passage from the upstream to the downstream of the air flow.

9. The air conditioning device according to claim 5,
wherein the outflow port is disposed on an upper side.

10. The air conditioning device according to claim 5,
wherein the passage is branched into the first passage and the second passage at a downstream position from the ion generator.
